(19) United States
(12) Patent Application Publication
(10) Pub. No.: US 2024/0161283 A1
(43) Pub. Date: May 16, 2024

(54) IMAGE PROCESSING DEVICE, IMAGE PROCESSING METHOD, AND STORAGE MEDIUM

(71) Applicant: NEC Corporation, Tokyo (JP)

(72) Inventor: Masahiro SAIKOU, Tokyo (JP)

(73) Assignee: NEC Corporation, Tokyo (JP)

(21) Appl. No.: 18/410,187

(22) Filed: Jan. 11, 2024

Related U.S. Application Data
(63) Continuation of application No. 18/574,109, filed on Jan. 1, 0001.

(30) Foreign Application Priority Data
Oct. 18, 2022 (WO) PCT/JP2022/038743

(51) Int. Cl. G06T 7/00; A61B 1/00; G06V 10/776; G16H 50/20

(52) U.S. Cl. CPC G06T 7/0012; A61B 1/000094; G06V 10/776; G16H 50/20; G06T 2207/10068; G06T 2207/20081; G06T 2207/30096

(57) ABSTRACT

The image processing device 1X includes an acquisition means 30X, an inference means 32X, and an integration means 33X. The acquisition means 30X acquires an endoscopic image obtained by photographing an examination target. The inference means 32X generates plural inference results regarding an attention region of the examination target in the endoscopic image, based on the endoscopic image. The integration means 33X integrates plural inference results.

This application is a Continuation of U.S. application Ser. No. 18/574,109 filed on Dec. 26, 2023, which is a National Stage Entry of PCT/JP2023/031838 filed on Aug. 31, 2023, which claims priority from PCT International Application PCT/JP2022/038743 filed on Oct. 18, 2022, the contents of all of which are incorporated herein by reference, in their entirety.

### TECHNICAL FIELD

The present disclosure relates to a technical field of an image processing device, an image processing method, and a storage medium for processing an image to be acquired in endoscopic examination.

### BACKGROUND

There is known an endoscopic examination system which displays a photographed image of a lumen of an organ. For example, Patent Literature 1 discloses an endoscopic examination system that detects a target region based on an endoscopic image and a target region detection threshold value to determine whether the target region is either a flat lesion or a torose lesion.

### CITATION LIST

### Patent Literature

Patent Literature 1: WO2019/146077

### SUMMARY

### Problem to be Solved

Generally, endoscopic images possibly include a wide variety of lesions, and the photographing environments of endoscopic images are also diverse, and thus there are cases where accurate detection of lesion regions is very difficult. Therefore, there are cases where the lesion region that is a candidate for biopsy is not consistent even among doctors.

In view of the above-described issue, it is therefore an example object of the present disclosure to provide an image processing device, an image processing method, and a storage medium capable of accurately detecting an attention region included in an endoscope image.

### Means for Solving the Problem

One mode of the image processing device is an image processing device including: an acquisition means configured to acquire an endoscopic image obtained by photographing an examination target; an inference means configured to generate plural inference results regarding an attention region of the examination target in the endoscopic image, based on the endoscopic image; and an integration means configured to integrate plural inference results.

One mode of the image processing method is an image processing method executed by a computer, the image processing method including:
acquiring an endoscopic image obtained by photographing an examination target;
generating plural inference results regarding an attention region of the examination target in the endoscopic image, based on the endoscopic image; and
integrating plural inference results.

One mode of the storage medium is a storage medium storing a program executed by a computer, the program causing the computer to:
acquire an endoscopic image obtained by photographing an examination target;
generate plural inference results regarding an attention region of the examination target in the endoscopic image, based on the endoscopic image; and
integrate plural inference results.

### Effect

An example advantage according to the present invention is to accurately detect an attention region included in an endoscope image.

### BRIEF DESCRIPTION OF THE DRAWINGS

FIG. 1 It illustrates a schematic configuration of an endoscopic examination system.

FIG. 2 It illustrates a hardware configuration of an image processing device.

FIG. 3 It is a diagram showing an outline of a lesion detection process performed by the image processing device in the first example embodiment.

FIG. 4 It illustrates an example of a functional block of the lesion detection process in the first example embodiment.

FIG. 5A illustrates a calculation example of the degree of similarity between a model input image and a representative image.

FIG. 5B illustrates a calculation example of the degree of similarity between a lesion reliability map of the model input image and the representative image.

FIG. 6 It illustrates an example a display screen image displayed by a display device in an endoscopic examination.

FIG. 7 It is an example of a flowchart showing an outline of a process performed by the image processing device during the endoscopic examination in the first example embodiment.

FIG. 8 It is a diagram showing an outline of a lesion detection process performed by the image processing device in a second example embodiment.

FIG. 9 It is a functional block diagram of the image processing device relating to the lesion detection process in the second example embodiment.

FIG. 10 It is an example of a flowchart showing an outline of a process performed by the image processing device during the endoscopic examination in the second example embodiment.

FIG. 11 It is a diagram showing an outline of the lesion detection process performed by the image processing device in a third example embodiment.

FIG. 12 It is an example of a flowchart showing an outline of a process performed by the image processing device during the endoscopic examination in the third example embodiment.

FIG. 13 It is a block diagram of an image processing device according to a fourth example embodiment.

FIG. 14 It is an example of a flowchart executed by the image processing device in the fourth example embodiment.

### EXAMPLE EMBODIMENTS

Hereinafter, example embodiments of an image processing device, an image processing method, and a storage medium will be described with reference to the drawings.

### First Example Embodiment

(1) System Configuration

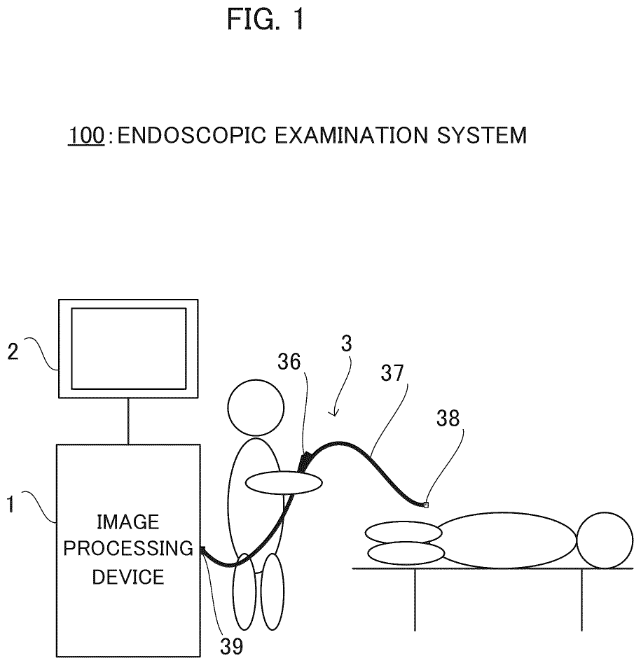

FIG. 1 shows a schematic configuration of an endoscopic examination system 100. As shown in FIG. 1, the endoscopic examination system 100 is a system that detects a part (also referred to as “lesion part”) of an examination target suspected of lesion and presents the part as a candidate part for cell collection (biology) to an examiner such as a doctor who conducts examination or treatment using an endoscope. Thereby, the endoscopic examination system 100 can support an examiner such as a doctor to perform decision making, such as determination of the biopsy part, manipulation of the endoscope, and determination of a treatment policy for the subject of the examination. The endoscopic examination system 100 mainly includes an image processing device 1, a display device 2, and an endoscope 3 connected to the image processing device 1.

The image processing device 1 acquires an image (also referred to as “endoscopic image Ia”) captured by the endoscope 3 in time series from the endoscope 3 and displays a screen image based on the endoscopic image Ia on the display device 2. The endoscopic image Ia is an image captured at a predetermined frame rate in at least one of the insertion process of the endoscope 3 to the subject and/or the ejection process of the endoscope 3 from the subject. In the present example embodiment, the image processing device 1 analyzes the endoscopic image Ia to detect a region (also referred to as “lesion region”) of the lesion part in the endoscopic image Ia, and displays the information on the detection result on the display device 2. The lesion region is an example of “attention region”.

The display device 2 is a display or the like for display information based on the display signal supplied from the image processing device 1.

The endoscope 3 mainly includes an operation unit 36 for examiner to perform a predetermined input, a shaft 37 which has flexibility and which is inserted into the organ to be photographed of the subject, a tip unit 38 having a built-in photographing unit such as an ultra-small image pickup device, and a connecting unit 39 for connecting with the image processing device 1.

The configuration of the endoscopic examination system 100 shown in FIG. 1 is an example, and various change may be applied thereto. For example, the image processing device 1 may be configured integrally with the display device 2. In another example, the image processing device 1 may be configured by a plurality of devices.

It is noted that the target of the endoscopic examination in the present disclosure may be any organ subject to endoscopic examination such as large bowel, esophagus, stomach, and pancreas. Examples of the target of the endoscopic examination in the present disclosure include a laryngendoscope, a bronchoscope, an upper digestive tube endoscope, a duodenum endoscope, a small bowel endoscope, a large bowel endoscope, a capsule endoscope, a thoracoscope, a laparoscope, a cystoscope, a cholangioscope, an arthroscope, a spinal endoscope, a blood vessel endoscope, and an epidural endoscope. A disorder (condition) of the lesion part subjected to detection in the endoscopic examination are exemplified as (a) to (f) below.
(a) Head and neck: pharyngeal cancer, malignant lymphoma, papilloma
(b) Esophagus: esophageal cancer, esophagitis, esophageal hiatal hernia, Barrett's esophagus, esophageal varices, esophageal achalasia, esophageal submucosal tumor, esophageal benign tumor
(c) Stomach: gastric cancer, gastritis, gastric ulcer, gastric polyp, gastric tumor
(d) Duodenum: duodenal cancer, duodenal ulcer, duodenitis, duodenal tumor, duodenal lymphoma
(e) Small bowel: small bowel cancer, small bowel neoplastic disease, small bowel inflammatory disease, small bowel vascular disease
(f) Large bowel: colorectal cancer, colorectal neoplastic disease, colorectal inflammatory disease; colorectal polyps, colorectal polyposis, Crohn's disease, colitis, intestinal tuberculosis, hemorrhoids.

(2) Hardware Configuration

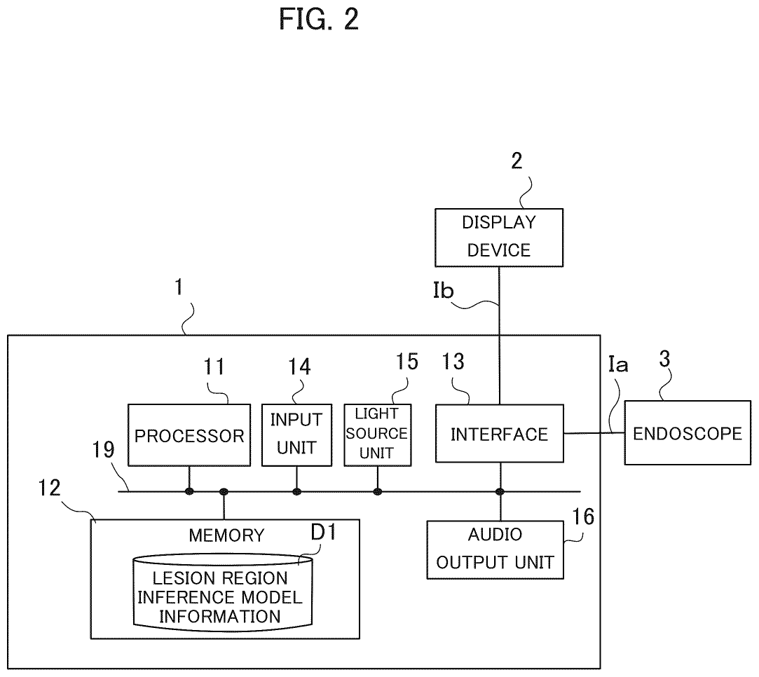

FIG. 2 shows a hardware configuration of the image processing device 1. The image processing device 1 mainly includes a processor 11, a memory 12, an interface 13, an input unit 14, a light source unit 15, and an audio output unit 16. Each of these elements is connected to one another via a data bus 19.

The processor 11 executes a predetermined process by executing a program or the like stored in the memory 12. The processor 11 is one or more processors such as a CPU (Central Processing Unit), a GPU (Graphics Processing Unit), and a TPU (Tensor Processing Unit). The processor 11 may be configured by a plurality of processors. The processor 11 is an example of a computer.

The memory 12 is configured by a variety of volatile memories which is used as working memories, and nonvolatile memories which stores information necessary for the process to be executed by the image processing device 1, such as a RAM (Random Access Memory) and a ROM (Read Only Memory). The memory 12 may include an external storage device such as a hard disk connected to or built in to the image processing device 1, or may include a storage medium such as a removable flash memory. The memory 12 stores a program for the image processing device 1 to execute each process in the present example embodiment.

The memory 12 also stores a lesion region inference model information D1 which is information regarding a lesion region inference model. The lesion region inference model is a machine learning model configured to generate an inference result regarding a lesion region to be detected in the endoscopic examination, and parameters required for the model are recorded in the lesion region inference model information D1. For example, the lesion region inference model is configured to output, when an endoscopic image is inputted thereto, an inference result indicating the lesion region in the inputted endoscopic image. The lesion region inference model may be a model (including a statistical model, hereinafter the same) that includes an architecture employed in any machine learning, such as a neural network and a support vector machine. Examples of the typical model of such a neural network include Fully Convolutional Network, SegNet, U-Net, V-Net, Feature Pyramid Network, Mask R-CNN, and DeepLab. When the lesion region inference model is configured by a neural network, the lesion region inference model informational D1 includes various parameters regarding the structure, the neuron structure of each layer, the number of filters and filter size in each layer, and the weight for each element of each filter, for example.

For example, the inference result outputted by the lesion region inference model is a map of scores (also referred to as “lesion reliability scores”) each indicating the degree of reliability of the presence of a lesion region for each unit region in the inputted endoscopic image. The above-described map is also referred to as “lesion reliability map”, hereinafter. For example, a lesion reliability map is an image showing a lesion reliability score per pixel (which may include sub-pixels) or per group of pixels. It is hereinafter assumed that the higher the lesion reliability score is, the higher the degree of reliability of the presence of the lesion region becomes. It is noted that the lesion reliability map may be a mask image showing the lesion region by two values (binary). Thus, the lesion region inference model is a model which learned the relation between an image inputted to the lesion region inference model and the lesion region in the inputted image.

The lesion region inference model is trained in advance on the basis of sets of an input image that confirms to the input format of the lesion region inference model and correct answer data (in this example embodiment, a correct lesion reliability map) indicating the correct inference result to be outputted by the lesion region inference model when the input image is inputted thereto. Then, parameters and the like of each model obtained through the training are stored in the memory 12 as the lesion region inference model information D1.

Further, the memory 12 may optionally include other information necessary for the image processing device 1 to perform each process in the present example embodiment.

The lesion region inference model information D1 may be stored in a storage device separate from the image processing device 1. In this instance, the image processing device 1 receives the lesion region inference model information D1 from the above-described storage device.

The interface 13 performs an interface operation between the image processing device 1 and an external device. For example, the interface 13 supplies the display information “Ib” generated by the processor 11 to the display device 2. Further, the interface 13 supplies the light generated by the light source unit 15 to the endoscope 3. The interface 13 also provides an electrical signal to the processor 11 indicative of the endoscopic image Ia supplied from the endoscope 3. The interface 13 may be a communication interface, such as a network adapter, for wired or wireless communication with the external device, or a hardware interface compliant with a USB (Universal Serial Bus), a SATA (Serial AT Attachment), or the like.

The input unit 14 generates an input signal based on the operation by the examiner. Examples of the input unit 14 include a button, a touch panel, a remote controller, and a voice input device. The light source unit 15 generates light for supplying to the pointed end unit 38 of the endoscope 3. The light source unit 15 may also incorporate a pump or the like for delivering water and air to be supplied to the endoscope 3. The audio output unit 16 outputs a sound under the control of the processor 11.

(3) Lesion Detection Process A description will be given of the lesion detection process that is a process related to detection of the lesion region. In summary, the image processing device 1 augments the endoscopic image Ia to “N” pieces of images (N is an integral number of 2 or more) by data augmentation, and integrates the inference results obtained by inputting each of the N pieces of images into the lesion region inference model. Thus, the image processing device 1 accurately detects the lesion region serving as a candidate part for biopsy.

(3-1) Overview

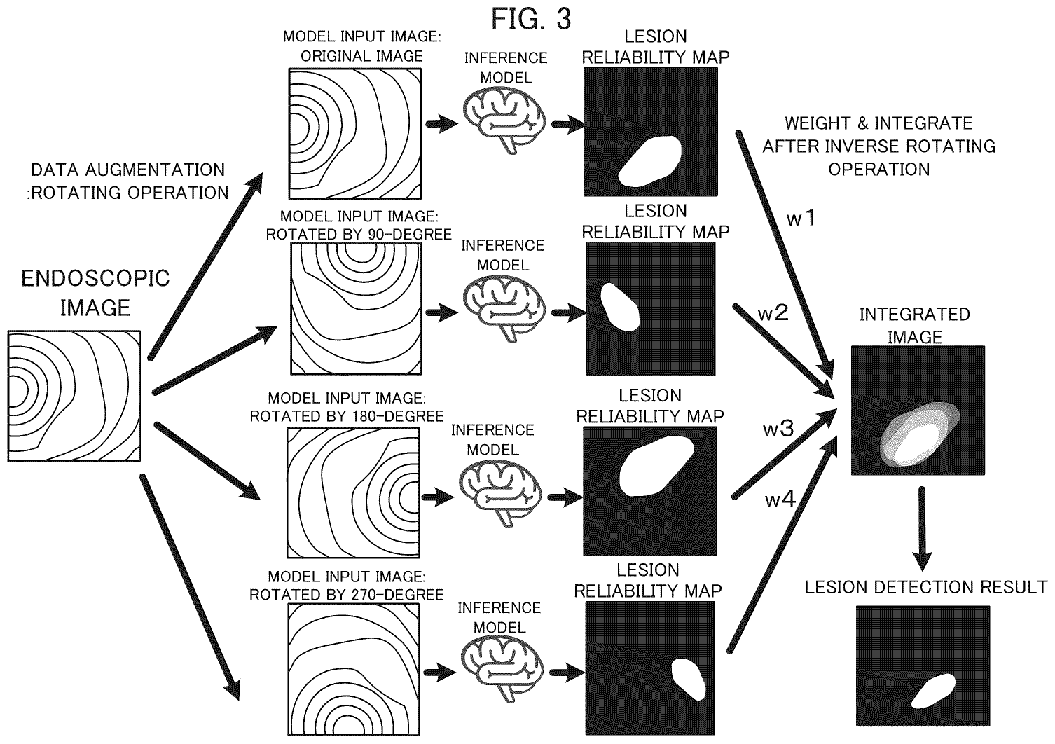

FIG. 3 is a diagram illustrating an outline of a lesion detection process that is executed by the image processing device 1 according to the first example embodiment.

First, the image processing device 1 generates, by data augmentation, N pieces of images from each endoscopic image Ia obtained from the endoscope 3 at a predetermined frame rate as images (also referred to as “model input images”) to be inputted to the lesion region inference model. In the example shown in FIG. 3, as an example, the image processing device 1 generates four (i.e., N=4) pieces of model input images by rotating the endoscopic image Ia clockwise by 0, 90, 180, and 270 degrees. Examples of data augmentation method to be adopted include not only the rotating operation but also any operation such as an image size changing operation, a brightness changing operation (including a designation of presence or absence of the normalization of the brightness), a color changing operation (including an adjustment of the strength or weakness of the red), and a combination thereof.

Next, the image processing device 1 inputs each model input image to the lesion region inference model and acquires a lesion reliability map that is an inference result regarding the lesion region outputted by the lesion region inference model. In FIG. 3, as an example, it is assumed that the lesion reliability map is a mask image in which a lesion region is indicated by a binary value (here, white is a lesion region).

Then, the image processing device 1 generates an image (also referred to as “integrated image”) into which the N (N=4) pieces of lesion reliability maps are integrated by weighted average. Here, the weighting factor “wi” (“i” is the index of the inference result, i=1, . . . , N) the sum of which is equal to 1 is used, and by averaging, for each pixel, the lesion reliability scores of the N lesion reliability maps weighted by the respective weighting factors, the image processing device 1 determines the lesion reliability score per pixel of the integrated image. In FIG. 3, it is assumed in the integrated image that the closer to white the color is, the higher the lesion reliability score (i.e., the degree of reliability that a lesion region is present) becomes. In addition, when geometric image conversion such as rotation of an image or enlargement/reduction of a size is performed as data augmentation, the images are integrated after restoration of the angle and the image size according to the original image. Therefore, in the example shown in FIG. 3, the image processing device 1 applies inverse (counterclockwise) rotating operations (i.e., rotating operations cancelling the rotating operations for generating the model input images) by 0-degree, 90-degrees, 180-degrees, and 270-degrees to the lesion reliability maps, respectively, and integrates the images after the application by weighed average.

Then, the image processing device 1 determines that pixels, in the integrated image, each having a pixel value indicating that the reliability score of the presence of the lesion region is equal to or larger than a predetermined degree constitutes the lesion region, and generates an image (here, a mask image indicating the lesion region) indicating the final lesion detection result. The image processing device 1 displays the mask image together with the endoscopic image Ia.

Here, generally, the endoscopic image Ia used for detecting a lesion possibly includes various types of lesions, and the photographing environment of the endoscopic image Ia is also diverse, and thus accurate detection of a lesion region could be very difficult. For example, there are types of lesions included in endoscopic image Ia, such as a torose lesion, a flat lesion, and a recessed lesion, and the shapes vary. In addition, the photographing environment differs depending on the position of lesion, lighting conditions, the presence or absence of water spray, and the presence or absence of blurring. Therefore, the lesion regions that are candidates for biopsy may not be consistent even among doctors.

Taking the above into consideration, the image processing device 1 generates a plurality of inference results and identifies the final lesion region by integrating the inference results. Thus, it is possible to suitably present a lesion region as a candidate for the biopsy point to the examiner.

(3-2) Functional Blocks

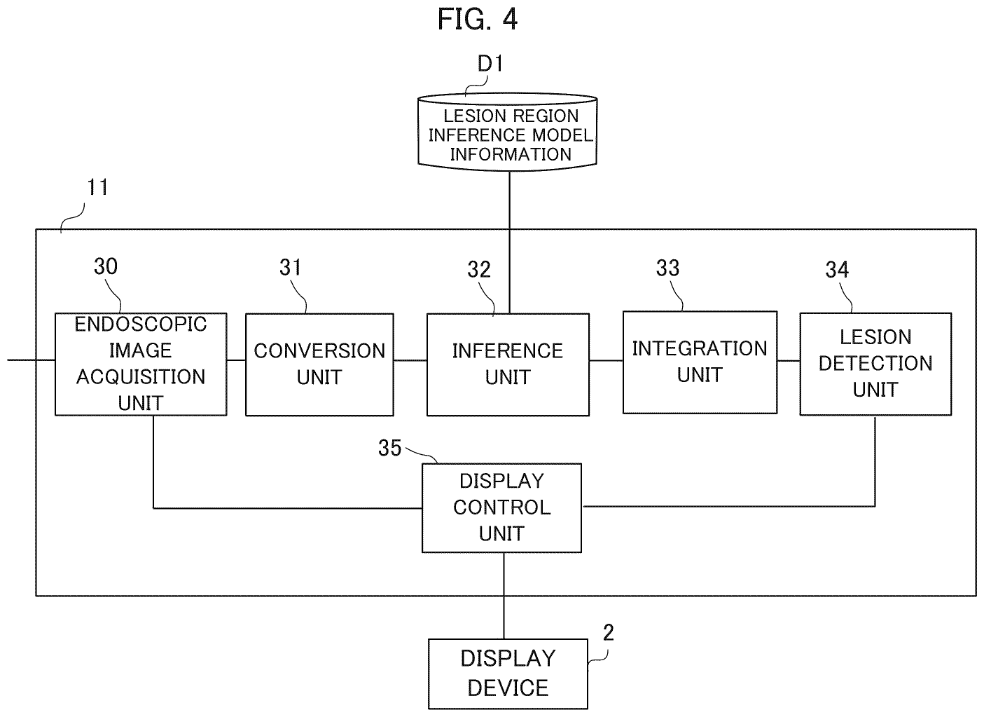

FIG. 4 is an example of a functional block of the lesion detection process in the first example embodiment. The processor 11 of the image processing device 1 functionally includes an endoscopic image acquisition unit 30, a conversion unit 31, an inference unit 32, an integration unit 33, a lesion detection unit 34, and a display control unit 35. In FIG. 4, any blocks to exchange data with each other are connected with each other by a solid line, but the combination of blocks to exchange data with each other is not limited thereto. The same applies to the drawings of other functional blocks described below.

The endoscopic image acquisition unit 30 acquires the endoscopic image Ia taken by the endoscope 3 through the interface 13 at predetermined intervals. The endoscopic image acquisition unit 30 supplies the acquired endoscopic image Ia to the conversion unit 31 and the display control unit 35, respectively. Then, at the time intervals in which the endoscopic image acquisition unit 30 acquires the endoscopic image Ia, the subsequent processing units periodically perform the processes described later. Thereafter, the time periodically identified at the above-mentioned intervals is also referred to as the “processing time”.

The conversion unit 31 generates N model input images from a single endoscopic image Ia by data augmentation. In this case, for example, the conversion unit 31 generates N pieces of model input images differing from each other, by applying rotating operations, image size changing operations, brightness changing operations, color changing operations, or any combination thereof to the endoscopic image Ia. The method of data augmentation is not limited to the operations exemplified above, but may be any operations to be used for data augmentation. The conversion unit 31 supplies the generated N pieces of model input images to the inference unit 32.

The inference unit 32 acquires N pieces of lesion reliability maps that are inference results regarding the lesion region on the basis of the N pieces of model input images and the lesion region inference model built by referring to the lesion region inference model information D1. In this case, the inference unit 32 inputs each of the N pieces of model input images to the lesion region inference model in order, and acquires the N pieces of lesion reliability maps outputted by the lesion region inference model. The inference unit 32 supplies the N pieces of lesion reliability maps to the integration unit 33.

The integration unit 33 generates an integrated image into which N pieces of the lesion reliability maps are integrated by weighted average. In this case, after converting the angle and the image size of each lesion reliability map so as to cancel the conversion operations applied by the conversion unit 31, the inference unit 32 sets the weighting factor wi (i=1, . . . , N) whose total value becomes 1 to each lesion reliability map. Then, for each pixel, the inference unit 32 sums up the lesion reliability scores of the N pieces of lesion reliability maps each multiplied by the corresponding weighting factor wi and thereby determines the lesion reliability score of each pixel of the integrated image to be the sum. The weighting factor wi is determined, for example, on the basis of the degree of similarity between the corresponding model input image or the lesion reliability map and an image (also referred to as “representative image”) representing the input image or the correct answer data used for training the lesion region inference model. In other instances, the weighting factor wi is set to a fixed value (i.e., “1/N”) regardless of the index i so that the weights are equal. The method of determining the weighting factor wi will be described later. The integration unit 33 supplies the generated integrated image to the lesion detection unit 34.

Based on the integrated image, the lesion detection unit 34 determines whether or not there is a lesion region and identifies the lesion region upon determining that there is a lesion region. In this case, for example, if the number of pixels, whose lesion reliability scores are equal to or larger than a predetermined threshold value in the integrated image, is equal to or larger than a predetermined number, the lesion detection unit 34 determines that there is a lesion region and then identifies the lesion region configured by the pixels, in the integrated image, having the lesion reliability score equal to or larger than the predetermined threshold value. In some embodiments, the lesion detection unit 34 clusters the pixels having the lesion reliability score equal to or larger than the predetermined threshold value so that adjacent pixels form a cluster, and then regards such a cluster having a predetermined number of or more pixels as a lesion region. The lesion detection unit 34 supplies the display control unit 35 with a lesion detection result that is information indicating the determination result regarding the presence or absence of a lesion region and the identified lesion region.

The display control unit 35 generates the display information Ib based on the latest endoscopic image Ia supplied from the endoscopic image acquisition unit 30 and the lesion detection result supplied from the lesion detection unit 34, then supplies the display information Ib to the display device 2 thereby to causes the display device 2 to display the latest endoscopic image Ia and the lesion detection result. In some embodiments, the display control unit 35 may control the audio output unit 16 to output, by audio, a warning sound or a voice guidance for notifying the user that a lesion part is detected.

Each component of the endoscopic image acquisition unit 30, the conversion unit 31, the inference unit 32, the integration unit 33, the lesion detection unit 34, and the display control unit can be realized, for example, by the processor 11 which executes a program. In addition, the necessary program may be recorded in any non-volatile storage medium and installed as necessary to realize the respective components. In addition, at least a part of these components is not limited to being realized by a software program and may be realized by any combination of hardware, firmware, and software. At least some of these components may also be implemented using user-programmable integrated circuitry, such as FPGA (Field-Programmable Gate Array) and microcontrollers. In this case, the integrated circuit may be used to realize a program for configuring each of the above-described components. Further, at least a part of the components may be configured by a ASSP (Application Specific Standard Produce), ASIC (Application Specific Integrated Circuit) and/or a quantum processor (quantum computer control chip). In this way, each component may be implemented by a variety of hardware. The above is true for other example embodiments to be described later. Further, each of these components may be realized by the collaboration of a plurality of computers, for example, using cloud computing technology.

(3-3) Example of Setting Weighting Factor

Next, an example of setting the weighting factor wi by the integration unit 33 when the weighting factor wi is set for each index i will be described. In this instance, the integration unit 33 determines the weighting factor wi on the basis of the degree of similarity between each model input image or its lesion reliability map and the representative image representing the image including the lesion region to be detected.

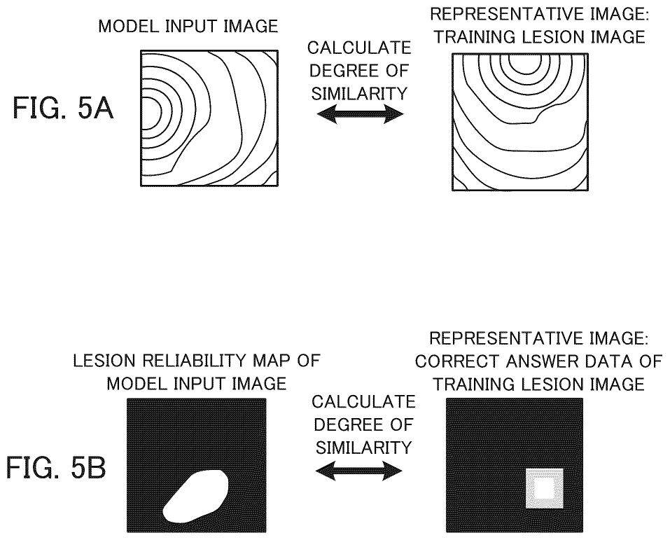

FIG. 5A shows an example of calculating the degree of similarity between the model input image with the index i and the representative image. In this example, the integration unit 33 uses, as a representative image, an endoscopic image (referred to as “training lesion image”) which includes a lesion region and which was used for training the lesion region inference model, and calculates the degree of similarity with the model input image for each index i.

In the example shown in FIG. 5A, an arbitrary one training lesion image is used as the representative image, but the representative image is not limited thereto. For example, the integration unit 33 may use, as the representative image, the average image of a plurality of training lesion images or an image integrated through any statistical method other than averaging. In another example, the integration unit 33 may respectively define a plurality of training lesion images as representative images, and uses, as the degree of similarity to determine the weighting factor wi, the average of the degrees of similarity between each of the training lesion images and the model input image with the index i.

As the degree of similarity between the model input image and the representative image, any index indicating the degree of similarity based on comparison between images may be calculated. Examples of such an index of the degree of similarity in this case include the correlation coefficient, the SSIM (Structural SIMilarity) index, the PSNR (Peak Signal-to-Noise Ratio) index, and the square error between corresponding pixels. In some embodiments, the integration unit 33 normalizes the model input image and the representative image in terms of the size and converts them into vectors. Then, the integration unit 33 calculates, as the degree of similarity, the cosine similarity of these vectors.

Then, the integration unit 33 calculates the degree of similarity described above for each index i, and sets the weighting factor wi so that the higher the degree of similarity for the index i is, the larger the weighting factor wi becomes. For example, when the degree of similarity for the index i is denoted by “Si”, the integration unit 33 sets the weighting factor wi according to the following equation using “Σ Si” representing the sum of the degrees of similarity Si.

Wi=Si/ΣSi

According to this example, it is possible to set the weighting factor wi so that the sum Σ wi for all index i is equal to 1 and the weighting factor wi increases with an increase in the corresponding degree of similarity Si.

FIG. 5B shows an example of calculating the degree of similarity between a lesion reliability map (in this case, a mask image) of the model input image with the index i and the representative image. In this example, the integration unit 33 uses, as the representative image, a correct answer (in this case, a mask image indicating a lesion region) of the lesion reliability map to be outputted by the lesion region inference model, wherein the correct answer is attached to the training lesion image through annotation. Then, the integration unit 33 calculates, for each index i, the degree of similarity between the correct answer of the lesion reliability map used for training and the lesion reliability map generated by the lesion region inference model from the model input image.

In the example shown in FIG. 5B, the representative image is set to the correct answer of the lesion reliability map for any single training lesion image. However, the representative image is not limited to the above example and may be set to an image obtained by averaging correct answers of plural training lesion images or an image obtained by integrating the correct answers through any statistical method other than averaging.

Then, the integration unit 33 calculates an arbitrary index indicating the degree of similarity based on the comparison between the images as the degree of similarity between the lesion reliability map of the training lesion image and the lesion reliability map of the model input image. Then, the integration unit 33 sets the weighting factor wi so that the sum E wi in all index i is equal to 1 and the weighting factor wi increases with an increase in the degree of similarity corresponding thereto. Specific examples of the method of calculating the degree of similarity and the method of setting the weighting factor wi based on the degree of similarity are identical to the examples shown in FIG. 5A.

(3-4) Display Example

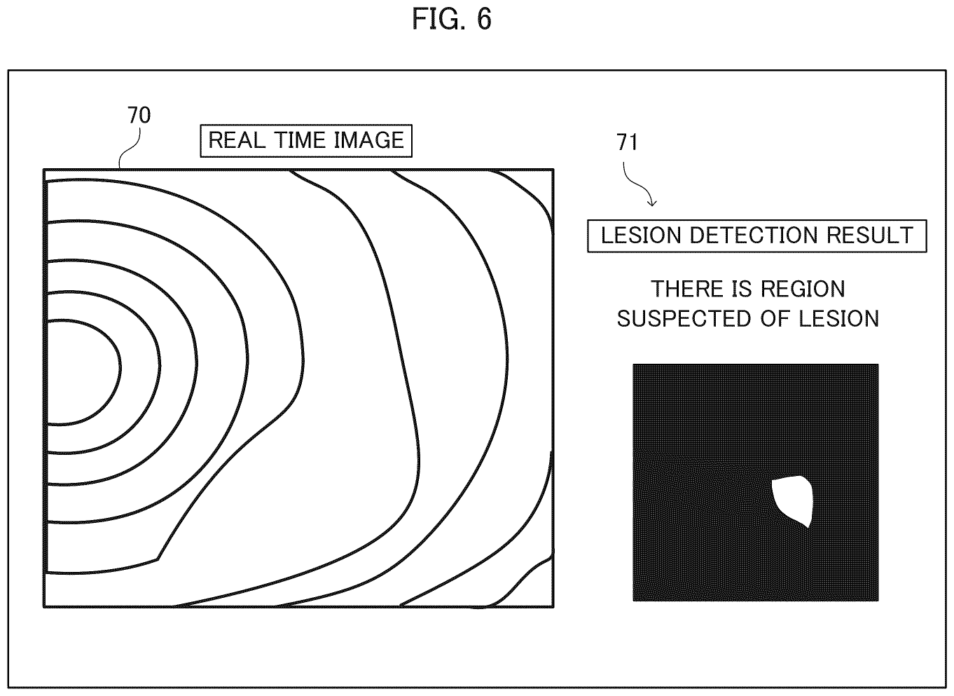

FIG. 6 shows an example of a display screen image displayed by the display device 2 in the endoscopic examination. The display control unit 35 of the image processing device 1 supplies the display device 2 with the display information Ib generated on the basis of the endoscopic image Ia acquired by the endoscopic image acquisition unit 30 and the lesion detection result generated by the lesion detection unit 34. The display control unit 35 transmits the endoscopic image Ia and the display information Ib to the display device 2 to display the above-described display screen image on the display device 2. In the display screen image example shown in FIG. 6, the display control unit 35 of the image processing device 1 provides a real-time image display area 70 and a lesion detection result display area 71 on the display screen image.

The display control unit 35 displays, in the real-time image display area 70, a moving image representing the latest endoscopic image Ia. Furthermore, in the lesion detection result display area 71, the display control unit 35 displays the lesion detection result generated by the lesion detection unit 34. Since the lesion detection unit 34 determines that the lesion part is present at the time of displaying the display screen image shown in FIG. 6, the display control unit displays, in the lesion detection result display area 71, a text message indicating that a lesion is likely to be present and a mask image indicating the lesion region on the basis of the lesion detection result. In some embodiments, instead of displaying a text message indicating that a lesion is likely to be present in the lesion detection result display area 71, or in addition to this, the display control unit 35 may output a sound (including voice) notifying the user that a lesion is likely to be present by the audio output unit 16.

Further, in some embodiments, the display control unit 35 may determine a coping method (remedy) based on a model and the lesion detection result of the examination target and output the coping method, wherein the model is generated through machine learning of a correspondence relation between a lesion detection result and the coping method. The method of determining the coping method is not limited to the method described above. Outputting such a coping method can further assist the examiner's decision making.

(3-5) Processing Flow

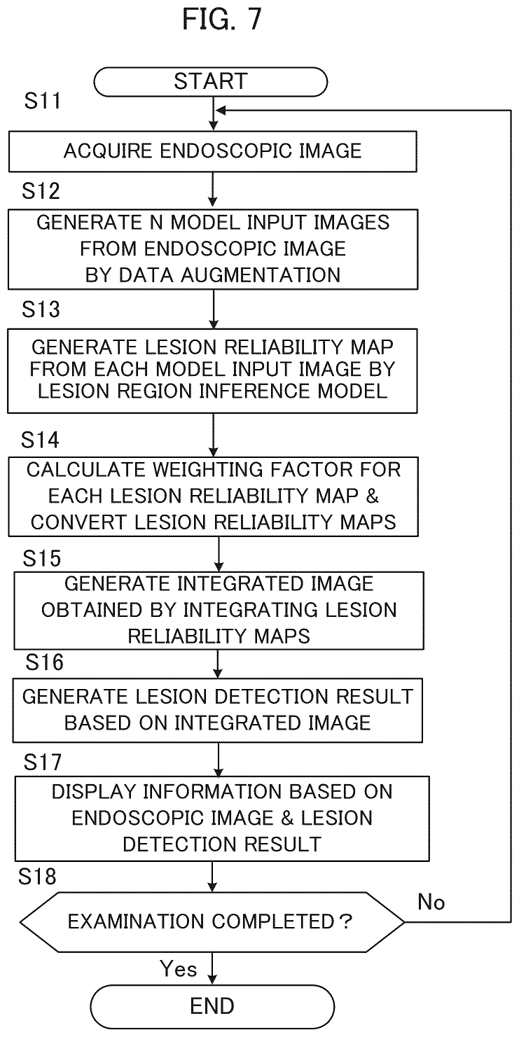

FIG. 7 is an example of a flowchart illustrating an outline of a process that is executed by the image processing device 1 during the endoscopic examination in the first example embodiment.

First, the image processing device 1 acquires an endoscopic image Ia (step S11). In this instance, the endoscopic image acquisition unit 30 of the image processing device 1 receives the endoscopic image Ia from the endoscope 3 through the interface 13.

Next, the image processing device 1 generates N pieces of model input images, which differ from each other, from the endoscopic image Ia acquired at step S11 by data augmentation (step S12). Then, the image processing device 1 generates a lesion reliability map from each model input image by using the lesion region inference model configured by referring to the lesion region inference model information D1 (step S13). In this case, the image processing device 1 acquires each lesion reliability map outputted from the lesion region inference model by inputting each model input image into the lesion region inference model.

Then, the image processing device 1 calculates the weighting factor wi for each lesion reliability map (step S14). In this case, for example, the image processing device 1 sets the weighting factor wi for each index i (i=1, . . . , N) on the basis of the degree of similarity between the model input image or the lesion reliability map and the corresponding representative image. In addition, the image processing device 1 applies the conversion operations regarding the angle and/or the size to the respective lesion reliability maps so as to cancel the conversion operations applied by data augmentation at step S12.

Next, the image processing device 1 generates an integrated image into which the lesion reliability maps are integrated using the corresponding weighting factors wi (step S15). Then, the image processing device 1 generates a lesion detection result based on the integrated image (step S16). Then, the image processing device 1 displays on the display device 2 the information based on the endoscopic image Ia obtained at step S11 and the lesion detection result generated at step S16 (step S17).

Then, the image processing device 1 determines whether or not the endoscopic examination has been completed after the process at step S17 (step S18). For example, the image processing device 1 determines that the endoscopic examination has been completed when a predetermined input or the like to the input unit 14 or the operation unit 36 is detected. Upon determining that the endoscopic examination has been completed (step S18; Yes), the image processing device 1 ends the process of the flowchart. On the other hand, upon determining that the endoscopic examination has not been completed (step S18; No), the image processing device 1 gets back to the process at step S11. Then, the image processing device 1 proceeds with the processes at step S11 to step S17 using the endoscopic image Ia newly generated by the endoscope 3.

(4) Modifications

After the examination, the image processing device 1 may process the video image configured by endoscopic images Ia generated in the endoscopic examination.

For example, when a video image to be processed is designated based on the user input by the input unit 14 at any timing after the examination, the image processing device 1 sequentially performs processing of the flowchart shown in FIG. 7 for the time series endoscopic images Ia constituting the video image. Then, the image processing device 1 terminates the process of the flowchart upon determining that the target video image has ended at step S18. In contrast, it gets back to the process at step S11 upon determining that the target video image has not ended, and proceeds with the process of the flowchart for the subsequent endoscopic image Ia in the time series.

The detection target is not limited to a lesion region, but may be an any region (also referred to as “attention region”) in the endoscopic imaging Ia that represents any attention point where the examiner needs attention. Examples of the attention part include a lesion part, a part with inflammation, a part with an operative scar or other cut part, a part with a fold or protrusion, and a part on the wall surface of the lumen where the tip unit 38 of the endoscope 3 tends to get contact (caught).

It is noted that this modification is also applied to the second example embodiment and the third example embodiment described later.

### Second Example Embodiment

The image processing device 1 according to the second example embodiment differs from the first example embodiment in that, instead of generating N pieces of lesion reliability maps from N pieces of model input images generated from an endoscopic image Ia, it generates N pieces of lesion reliability maps from an endoscopic image Ia using N pieces of different lesion region inference models. Hereinafter, the same components as in the first example embodiment are appropriately denoted by the same reference numerals, and a description thereof will be omitted. It is hereinafter assumed that the hardware configuration of the image processing device 1 according to the second example embodiment is identical to the hardware configuration shown in FIG. 2 described in the first example embodiment.

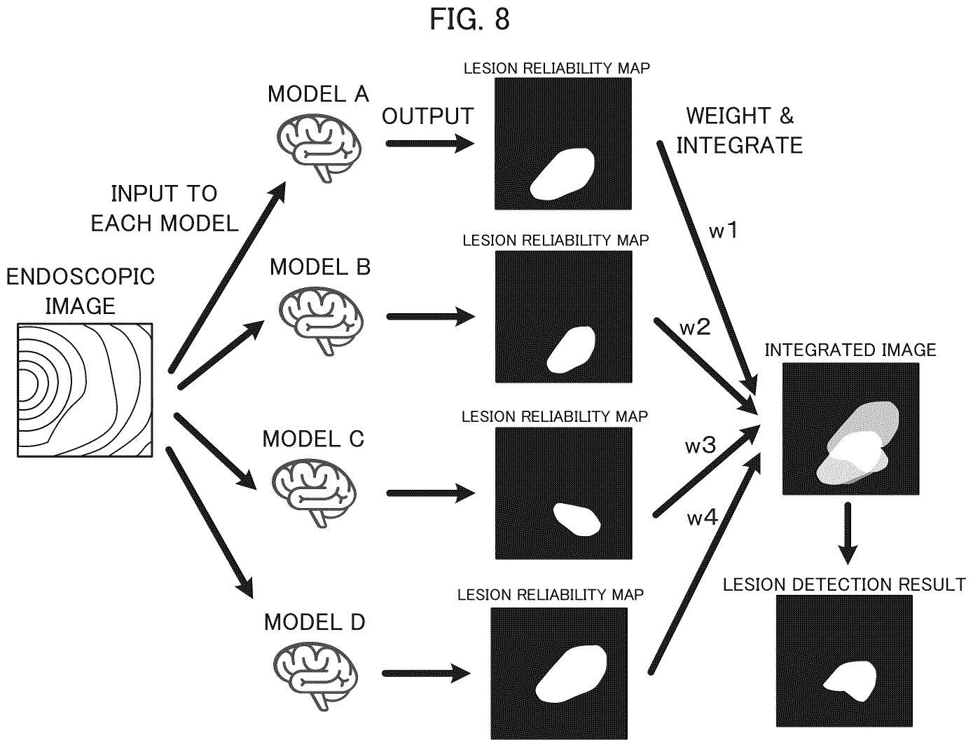

FIG. 8 is a diagram illustrating an outline of a lesion detection process that is executed by the image processing device 1 according to the second example embodiment.

First, the image processing device 1 inputs each endoscopic image Ia obtained from the endoscope 3 at a predetermined frame rate into N pieces of lesion region inference models (models A to D, in this case). Thus, the image processing device 1 acquires N pieces of lesion reliability maps from the N pieces of lesion region inference models.

It is noted that the N pieces of lesion region inference models differ from one another in at least one of the architecture and/or the training data used for training. Thus, the N pieces of lesion inference models generate different inference results even when the same endoscopic image Ia is inputted thereto.

In a case of a deep learning model, examples of difference in the architecture include difference in at least one of the layer structure, the neuron structure of each layer, the number of filters and the size of filters in each layer, and/or the weight for each element of each filter. In some embodiments, the N pieces of lesion region inference models may include models (e.g., models based on a support vector machine) other than deep learning models or combinations of deep learning models and models other than deep learning models.

According to an example of the difference in the training data, training datasets (i.e., training datasets corresponding to N venders) that include endoscopic images and the corresponding correct answer data regarding lesion regions are prepared for respective venders and N pieces of lesion region inference model are trained by use of the training datasets for respective venders. According to another example of the difference in the training data, training datasets (i.e., training datasets corresponding to N lesion types) that include endoscopic images and the corresponding correct answer data regarding lesion regions are prepared for respective lesion types (e.g., a torose lesion, a flat lesion, and a recessed lesion) and N pieces of lesion region inference model are trained by use of the training datasets for respective lesion types.

Then, the image processing device 1 generates an integrated image into which the N pieces of the lesion reliability maps are integrated by using the corresponding weighting factors wi. In this case, the image processing device 1 sums up, for each pixel, the lesion reliability scores of the N pieces of the lesion reliability maps multiplied by the corresponding weighting factors and sets the sum as the lesion reliability score for the each pixel of the integrated image.

Then, the image processing device 1 determines that pixels in the integrated image, having the lesion reliability score indicating that the degree of reliability of the presence of the lesion region is equal to or larger than a predetermined degree, falls under a lesion region. Then, the image processing device 1 generates an image (a mask image indicative of the lesion region herein) indicative of the final lesion detection result. The image processing device 1 displays the mask image together with the endoscopic image Ia.

Thus, the image processing device 1 in the second example embodiment generates a plurality of inference results and finally identifies the lesion region by integrating the inference results. Thus, it becomes possible to suitably present a lesion region as a candidate for biopsy to the examiner.

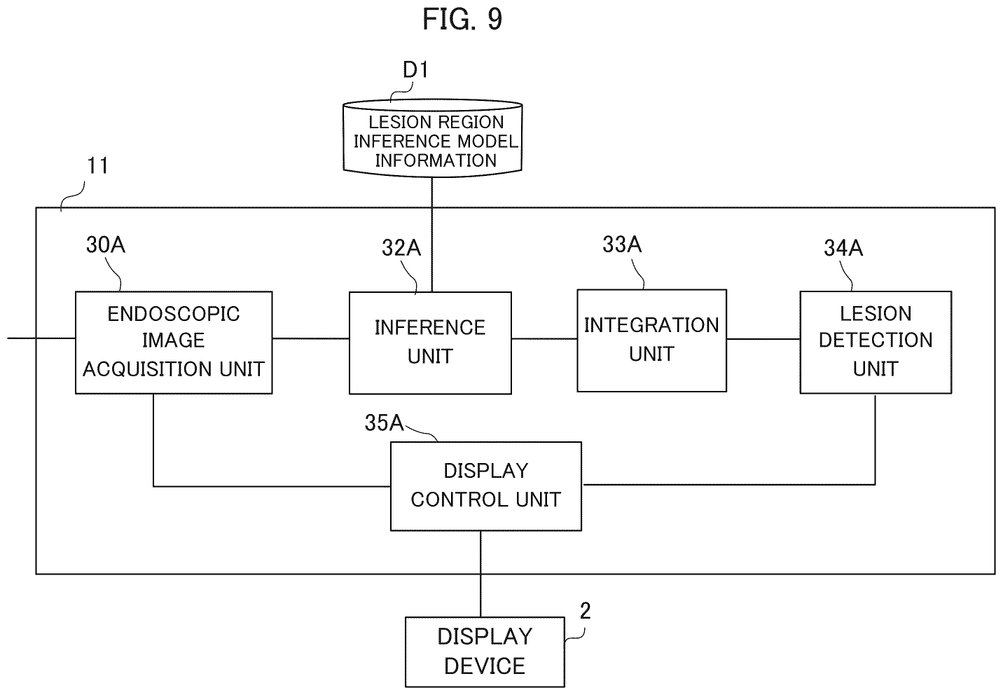

FIG. 9 is a functional block diagram of the image processing device 1 related to the lesion detection process in the second example embodiment. The processor 11 of the image processing device 1 according to the second example embodiment functionally includes an endoscopic image acquisition unit 30A, an inference unit 32A, an integration unit 33A, a lesion detection unit 34A, and a display control unit 35A. In addition, the memory 12 stores lesion region inference model information D1 which at least includes learned parameters of N pieces of lesion region inference models.

The endoscopic image acquisition unit 30A acquires the endoscopic image Ia captured by the endoscope 3 through the interface 13 at predetermined intervals. The endoscopic image acquisition unit 30A supplies the acquired endoscopic image Ia to the inference unit 32A and the display control unit 35A, respectively.

The inference unit 32A acquires N pieces of lesion reliability maps, which are inference results regarding a lesion region, based on the endoscopic image Ia and N pieces of lesion region inference models built by referring to the lesion region inference model information D1. In this case, the inference unit 32A inputs the endoscopic image Ia into N pieces of lesion region inference models, respectively, and acquires N pieces of lesion reliability maps outputted by the lesion region inference models in response to the input thereto. The inference unit 32A supplies N pieces of lesion reliability maps to the integration unit 33A.

The integration unit 33A generates an integrated image by integrating N pieces of lesion reliability maps by weighted average. For example, the integration unit 33A sets each weighting factor wi to an equal value (i.e., “1/N”) regardless of the index i. In another example, the integration unit 33A sets each weighting factor wi based on the degree of similarity between the lesion reliability map for each index i and the representative image. In this case, for example, the representative image is a correct answer of the lesion reliability map used for training the lesion region inference model corresponding to the index i. It is noted that examples of the “correct answer of the lesion reliability map” include an image obtained by averaging lesion reliability maps indicated by correct answer data corresponding to plural training lesion images, and an image obtained by integrating the above-mentioned lesion reliability maps through any statistical method other than the average. Thus, the representative image may be prepared in advance according to the training data used for the lesion region inference model corresponding to each index i.

Based on the integrated image generated by the integration unit 33A, the lesion detection unit 34A determines whether or not there is a lesion region and identifies the lesion region if the lesion region is determined to be present. Then, the lesion detection unit 34A supplies the determination result that is information indicative of the presence or absence of the lesion region and the identified lesion region to the display control unit 35A. The process performed by the lesion detection unit 34A is identical to the process performed by the lesion detection unit 34.

The display control unit 35A generates the display information Ib based on the latest endoscopic image Ia supplied from the endoscopic image acquisition unit 30A and the lesion detection result supplied from the lesion detection unit 34A. Then, the display control unit 35A supplies the display information Ib to the display device 2 thereby to cause the display device 2 to display the latest endoscopic image Ia and the lesion detection result. The process executed by the display control unit 35A is identical to the process executed by the display control unit 35.

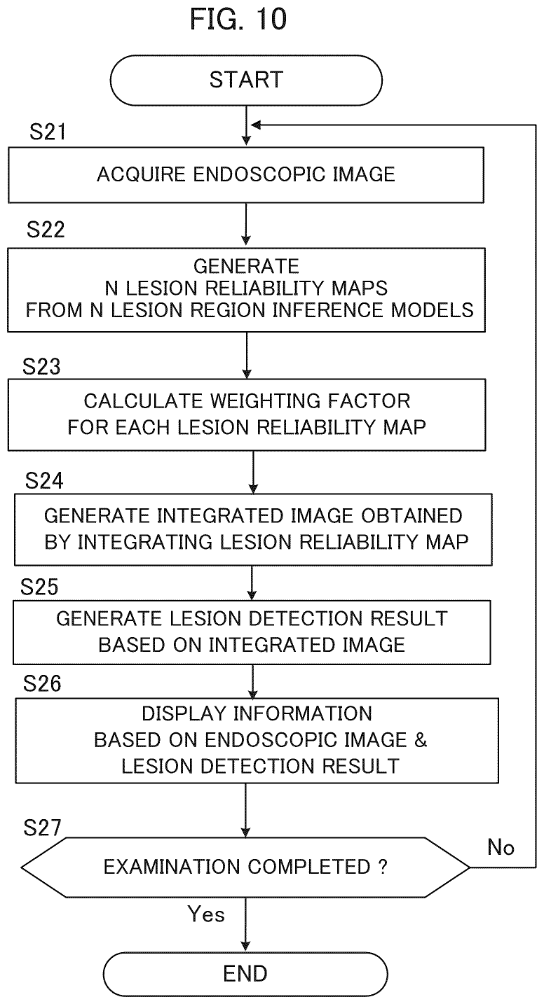

FIG. 10 is an example of a flowchart illustrating an outline of a process that is executed by the image processing device 1 during the endoscopic examination in the second example embodiment.

First, the image processing device 1 acquires an endoscopic image Ia (step S21). Next, the image processing device 1 generates N pieces of lesion reliability maps from the endoscopic image Ia acquired at step S11 by using the N pieces of lesion region inference models configured by referring to the lesion region inference model information D1 (step S22). In this instance, the image processing device 1 acquires the lesion reliability maps outputted from the lesion region inference models by inputting the endoscopic image Ia into the lesion region inference models, respectively.

Then, the image processing device 1 calculates the weighting factor wi for each lesion reliability map (step S23). In this instance, for example, the image processing device 1 sets each weighting factor wi on the basis of the similarity between the representative image prepared for each index i (i=1, . . . , N) and the lesion reliability map corresponding to the index i.

Next, the image processing device 1 generates an integrated image into which the lesion reliability maps are integrated by using the weighting factors wi (step S24). Then, the image processing device 1, based on the integrated image, generates a lesion detection result (step S25). Then, the image processing device 1 displays the information on the display device 2 based on the endoscopic image Ia obtained at step S11 and the lesion detection result generated at step S25 (step S26).

Then, the image processing device 1 determines whether or not the endoscopic examination has been completed after the process at step S26 (step S27). Upon determining that the endoscopic examination has been completed (step S27; Yes), the image processing device 1 ends the process of the flowchart. On the other hand, upon determining that the endoscopic examination has not been completed (step S27; No), the image processing device 1 gets back to the process at step S21. Then, the image processing device 1 proceeds with the processes at step S21 to step S26 using the endoscopic image Ia newly generated by the endoscope 3.

### Third Example Embodiment

The image processing device 1 according to the third example embodiment differs from the first example embodiment or the second example embodiment in that N different patterns (N patterns) of the setting condition are applied to one lesion region inference model to generate N pieces of lesion reliability maps from an endoscopic image Ia. Hereinafter, the same components as those in the first example embodiment or the second example embodiment are appropriately denoted by the same reference numerals, and a description thereof will be omitted.

The hardware configuration of the image processing device 1 according to the third example embodiment is the same as that shown in FIG. 2 described in the first example embodiment. The functional block of the image processing device 1 related to the lesion detection process in the third example embodiment is, for example, the same as the configuration shown in FIG. 9 described in the second example embodiment.

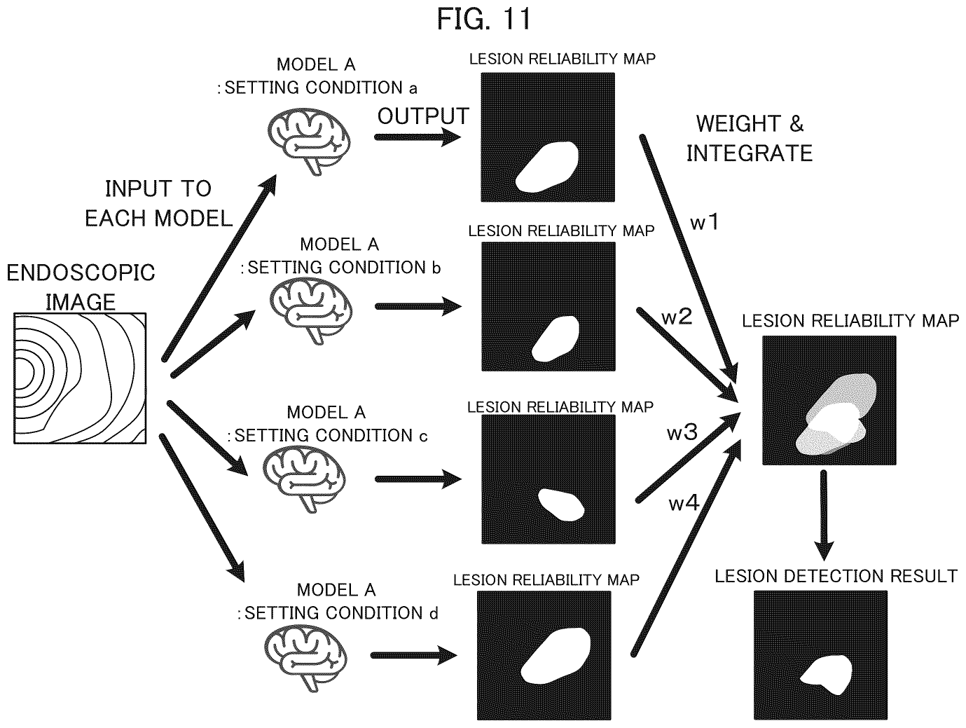

FIG. 11 is a diagram illustrating an outline of a lesion detection process that is executed by the image processing device 1 according to the third example embodiment.

First, the image processing device 1 inputs the respective endoscopic image Ia obtained from the endoscope 3 by a predetermined frame rate to a lesion region inference model (model A in this case) to which N patterns of the setting condition (setting conditions a to d in this case) are applied. Thus, the image processing device 1 acquires N pieces of lesion reliability maps in total from the lesion region inference model to which N patterns of the setting condition are respectively applied. In other words, the image processing device 1 inputs the endoscopic image Ia obtained at each processing time into the lesion region inference model by N times while changing the setting conditions of the lesion region inference model, and acquires N pieces of inference results outputted from the lesion region inference model.

For example, the setting condition herein is setting parameter (hyperparameter) of the lesion region inference model that can be adjusted by the user through input, and may be a threshold parameter for determining whether or not each pixel falls under a lesion region according to the lesion reliability score of the each pixel. Specifically, provided that the lesion reliability score ranges from 0 to 1 (for 1, the pixel is most likely to be a legion), the lesion reliability score of a certain pixel is set to 0 when the lesion reliability score of the certain pixel is smaller than the threshold parameter. This allows the certain pixel to be a non-lesion region. In this case, for example, if the threshold parameter is set to a value close to 1, only the region inferred by the inference model to be more likely to be lesion is regarded as the lesion region, and the other regions are set as the non-lesion region. In contrast, when the threshold parameter is set to a value close to 0, the region inferred by the inference model to be not a lesion could be also set as the lesion region. The setting in the former case is a setting (i.e., setting for prioritizing the precision) in which correct inference as a lesion region is prioritized and which is intended not to infer that a non-lesion region is a lesion region by mistake, whereas the setting in the latter case is a setting (i.e., setting for prioritizing the recall) intended to forbid a lesion region from being undetected while permitting a non-lesion region is included as a lesion region. By using setting parameters of the lesion region inference model with different intensions (which is difference in that the recall is prioritized or the precision is prioritized) in this way, each reliability map can be generated.

Then, the image processing device 1 generates an integrated image obtained by integrating the N pieces of lesion reliability maps while weighting them according to the weighting factors wi. In this case, the image processing device 1 determines each pixel value of the integrated image to be the sum, per pixel, of the lesion reliability scores of N pieces of lesion reliability maps multiplied by the corresponding weighing factors.

Then, the image processing device 1 determines that pixels in the integrated image, having the lesion reliability score indicating that the degree of reliability of the presence of the lesion region is equal to or larger than a predetermined degree, fall under a lesion region, and generates an image (here, a mask image indicating the lesion region) indicating the final lesion detection result. The image processing device 1 displays the mask image together with the endoscopic image Ia.

Thus, the image processing device 1 in the third example embodiment generates a plurality of inference results and identifies the final lesion region by integrating the inference results. Thus, it becomes possible to suitably present a lesion region as a candidate for biopsy to the examiner.

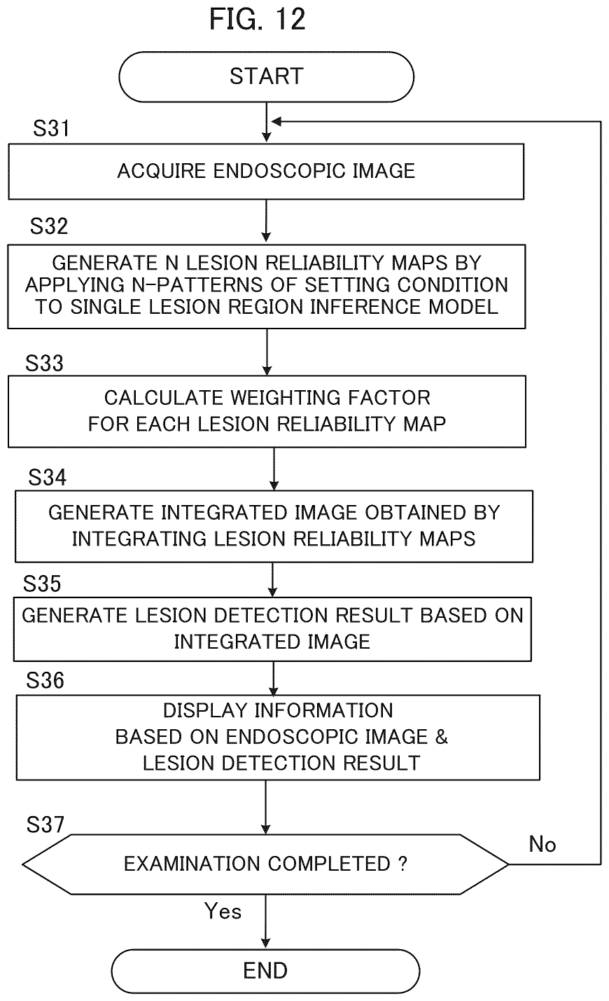

FIG. 12 is an example of a flowchart illustrating an outline of a process that is executed by the image processing device 1 during the endoscopic examination in the third example embodiment.

First, the image processing device 1 acquires the endoscopic image Ia (step S31). Next, the image processing device 1 applies N patterns of the setting condition to a single lesion region inference model configured by referring to the lesion region inference model information D1, and generates N pieces of lesion reliability maps from the endoscopic image Ia acquired at step S11 (step S32). In this case, the image processing device 1 inputs the endoscopic image Ia obtained at each processing time into the lesion region inference model by N times while changing the setting conditions of the lesion region inference model, and acquires N pieces of lesion reliability maps (i.e., inference results) outputted from the lesion region inference model.

Then, the image processing device 1 calculates the weighting factor wi for each lesion reliability map (step S33). In this case, for example, the image processing device 1 sets the weighting factor wi on the basis of the degree of similarity between the representative image common to all index i and the lesion reliability map corresponding to the index i.

Next, the image processing device 1 generates an integrated image into which the lesion reliability map is integrated using the weighting factors wi (step S34). Then, the image processing device 1 generates the lesion detection result based on the integrated image (step S35). Then, the image processing device 1 displays on the display device 2 the information based on the endoscopic image Ia obtained at step S11 and the lesion detection result generated at step S25 (step S36).

Then, the image processing device 1 determines whether or not the endoscopic examination has been completed after the process at step S36 (step S37). Upon determining that the endoscopic examination has been completed (step S37; Yes), the image processing device 1 ends the process of the flowchart. On the other hand, upon determining that the endoscopic examination has not been completed (step S37; No), the image processing device 1 gets back to the process at step S31. Then, the image processing device 1 proceeds with the processes at step S31 to step S36 using the endoscopic image Ia newly generated by the endoscope 3.

### Fourth Example Embodiment

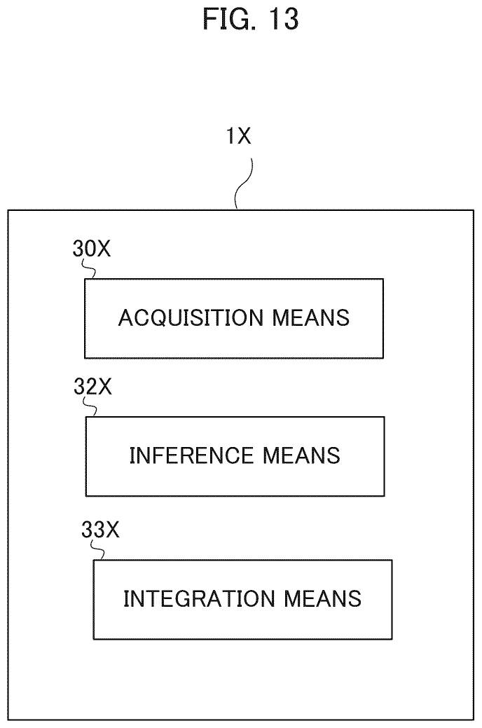

FIG. 13 is a block diagram of an image processing device 1X according to a fourth example embodiment. The image processing device 1X includes an acquisition means 30X, an inference means 32X, and an integration means 33X. The image processing device 1X may be configured by a plurality of devices.

The acquisition means 30X is configured to acquire an endoscopic image obtained by photographing an examination target. Examples of the acquisition means 30X include the endoscopic image acquisition unit 30 in the first example embodiment and the endoscopic image acquisition unit 30A in the second example embodiment or the third example embodiment. The acquisition means 30X may immediately acquire the endoscopic image generated by the photographing unit, or may acquire, at a predetermined timing, the endoscopic image stored in the storage device generated in advance by the photographing unit.

The inference means 32X is configured to generate plural inference results regarding an attention region of the examination target in the endoscopic image, based on the endoscopic image. Examples of the inference means 32X include the inference unit 32 in the first example embodiment and the inference unit 32A in the second example embodiment or the third example embodiment.

The integration means 33X is configured to integrate plural inference results. Examples of the integration means 33X include the integration unit 33 in the first example embodiment and the integration unit 33A in the second example embodiment or the third example embodiment.

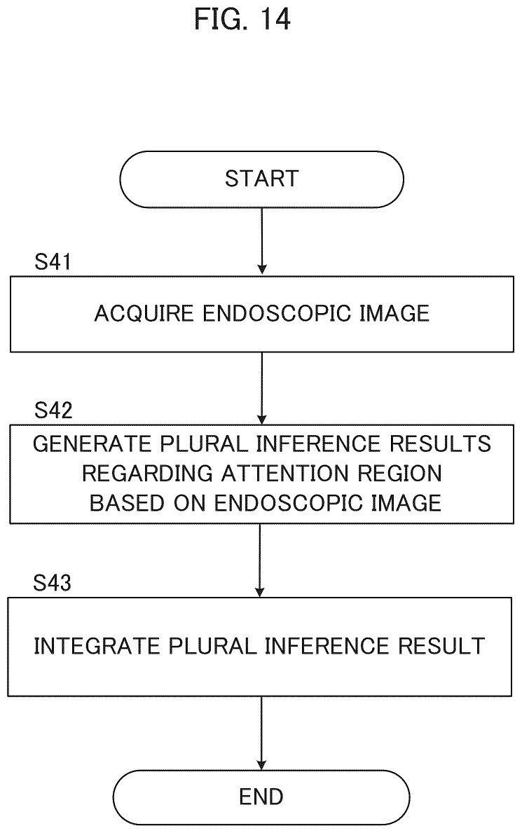

FIG. 14 is an example of a flowchart showing a processing procedure in the fourth example embodiment. The acquisition means 30X acquires an endoscopic image obtained by photographing an examination target (step S41). The inference means 32X generates plural inference results regarding an attention region of the examination target in the endoscopic image, based on the endoscopic image (step S42). The integration means 33X integrates plural inference results (step S43).

According to the fourth example embodiment, the image processing device 1X can accurately detect a region of an attention part from an endoscopic image obtained by photographing an examination target.

In the example embodiments described above, the program is stored by any type of anon-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments described above (including modifications, the same applies hereinafter) can be described as, but not limited to, the following Supplementary Notes.

[Supplementary Note 1]

An image processing device comprising:
an acquisition means configured to acquire an endoscopic image obtained by photographing an examination target;
an inference means configured to generate plural inference results regarding an attention region of the examination target in the endoscopic image, based on the endoscopic image; and
an integration means configured to integrate plural inference results.

[Supplementary Note 2]

The image processing device according to Supplementary Note 1, further comprising
a conversion means configured to convert the endoscopic image into plural images by data augmentation,
wherein the inference means is configured to generate an inference result regarding the attention region from each of the plural images.

[Supplementary Note 3]

The image processing device according to Supplementary Note 2,
wherein the inference means is configured to acquire the inference result outputted from an inference model by inputting each of the plural images into the inference model, and
wherein the inference model is a model obtained through machine learning of a relation between an image to be inputted to the inference model and the attention region in the image.

[Supplementary Note 4]

The image processing device according to Supplementary Note 1,
wherein the inference means is configured to acquire the plural inference results outputted from plural inference models by inputting the endoscopic image into the plural inference models, and
wherein the plural inference models each is a model obtained through machine learning of a relation between an image to be inputted to the model and the attention region in the image.

[Supplementary Note 5]

The image processing device according to Supplementary Note 4,
wherein the plural inference models are models such that at least one of architectures of the models and/or training data used for machine learning of the models are different from one another.

[Supplementary Note 6]

The image processing device according to Supplementary Note 1,
wherein the inference means is configured to acquire the plural inference results outputted from an inference model by inputting the endoscopic image into the inference model by plural times while changing setting conditions of the inference model, and
wherein the inference model is a model obtained through machine learning of a relation between an image to be inputted to the model and the attention region in the image.

[Supplementary Note 7]

The image processing device according to Supplementary Note 6,
wherein the setting condition is a threshold parameter for determining whether or not the attention region is present.

[Supplementary Note 8]

The image processing device according to Supplementary Note 7,
wherein the inference means is configured to at least acquire the inference results obtained from the inference model when the threshold parameter in which a recall is prioritized and the threshold parameter in which a precision is prioritized are respectively set to the inference model.

[Supplementary Note 9]

The image processing device according to Supplementary Note 3,
wherein the integration means is configured to integrate the plural inferences result while weighting each of the plural inference results based on a degree of similarity between
   each of the plural images and
   a training image, used for machine learning of the inference model, in which the attention region is included.

[Supplementary Note 10]

The image processing device according to any one of Supplementary Notes 3 to 8,
wherein the integration means is configured to integrate the plural inference result while weighting each of the plural inference results based on a degree of similarity between
   each of the plural inference results and
   correct answer data used for machine learning of the inference model.

[Supplementary Note 11]

The image processing device according to Supplementary Note 1, further comprising a detection means configured to detect the attention region based on an image into which the plural inference results are integrated.

[Supplementary Note 12]

The image processing device according to Supplementary Note 9, further comprising
an output control means configured to display or output, by audio, information regarding a result of the detection.

[Supplementary Note 13]

The image processing device according to Supplementary Note 12,
wherein the output control means is configured to output information regarding the result of the detection to assist examiner's decision making.

[Supplementary Note 14]

An image processing method executed by a computer, the image processing method comprising:
acquiring an endoscopic image obtained by photographing an examination target;
generating plural inference results regarding an attention region of the examination target in the endoscopic image, based on the endoscopic image; and
integrating plural inference results.

[Supplementary Note 15]

A storage medium storing a program executed by a computer, the program causing the computer to:
acquire an endoscopic image obtained by photographing an examination target;
generate plural inference results regarding an attention region of the examination target in the endoscopic image, based on the endoscopic image; and
integrate plural inference results.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All patent and Non-patent Literatures mentioned in this specification are incorporated by reference in its entirety.

### DESCRIPTION OF REFERENCE NUMERALS

1, 1X Image processing device
2 Display device
3 Endoscope
11 Processor
12 Memory
13 Interface
14 Input unit
15 Light source unit
16 Audio output unit
100 Endoscopic examination system

What is claimed is:

1. An image processing device comprising:
at least one memory configured to store instructions; and
at least one processor configured to execute the instructions to:
acquire an endoscopic image obtained by photographing an examination target;
generate plural inference results regarding an attention lesion of the examination target in the endoscopic image, based on the endoscopic image integrate plural inference results;
detect the lesion region based on an image into which the plural inference results are integrated; and
display the lesion region with color brightness depending on lesion reliability, as information regarding a result of the detection.

2. The image processing device according to claim 1,
is wherein the at least one processor is configured to further execute the instructions to convert the endoscopic image into plural images by data augmentation, and
wherein the at least one processor is configured to execute the instructions to generate an inference result regarding the attention region from each of the plural images.

3. The image processing device according to claim 2,
wherein the at least one processor is configured to execute the instructions to acquire the inference result outputted from an inference model by inputting each of the plural images into the inference model, and
wherein the inference model is a model obtained through machine learning of a relation between an image to be inputted to the inference model and the attention region in the image.

4. The image processing device according to claim 1,
wherein the at least one processor is configured to execute the instructions to acquire the plural inference results outputted from plural inference models by inputting the endoscopic image into the plural inference models, and
wherein the plural inference models each is a model obtained through machine learning of a relation between an image to be inputted to the model and the attention region in the image.

5. The image processing device according to claim 4,
wherein the plural inference models are models such that at least one of architectures of the models and/or training data used for machine learning of the models are different from one another.

6. The image processing device according to claim 1,
wherein the at least one processor is configured to execute the instructions to acquire the plural inference results outputted from an inference model by inputting the endoscopic image into the inference model by plural times while changing setting conditions of the inference model, and
wherein the inference model is a model obtained through machine learning of a relation between an image to be inputted to the model and the attention region in the image.

7. The image processing device according to claim 6,
wherein the setting condition is a threshold parameter for determining whether or not the attention region is present.

8. The image processing device according to claim 7,
wherein the at least one processor is configured to execute the instructions to at least acquire the inference results obtained from the inference model when the threshold parameter in which a recall is prioritized and the threshold parameter in which a precision is prioritized are respectively set to the inference model.

9. The image processing device according to claim 3,
wherein the at least one processor is configured to execute the instructions to integrate the plural inference results while weighting each of the plural inference results based on a degree of similarity between
each of the plural images and
a training image, used for machine learning of the inference model, in which the attention region is included.

10. The image processing device according to claim 3,
wherein the at least one processor is configured to execute the instructions to integrate the plural inference result while weighting each of the plural inference results based on a degree of similarity between
each of the plural inference results and
correct answer data used for machine learning of the inference model.

11. The image processing device according to claim 1,
wherein the at least one processor is configured to execute the instructions to output information regarding the result of the detection to assist examiner's decision making.

12. An image processing method executed by a computer, the image processing method comprising:
acquiring an endoscopic image obtained by photographing an examination target;
generating plural inference results regarding an attention region of the examination target in the endoscopic image, based on the endoscopic image; and
integrating plural inference results detecting the lesion region based on an image into which the plural inference results are integrated; and
displaying the lesion region with color brightness depending on lesion reliability, as information regarding a result of the detection.

13. A non-transitory computer readable storage medium storing a program executed by a computer, the program causing the computer to:
acquire an endoscopic image obtained by photographing an examination target;
generate plural inference results regarding an attention region of the examination target in the endoscopic image, based on the endoscopic image; and
integrate plural inference results detect the lesion region based on an image into which the plural inference results are integrated; and
display the lesion region with color brightness depending on lesion reliability, as information regarding a result of the detection.
